(19) United States
(12) Patent Application Publication
(10) Pub. No.: US 2012/0298099 A1
(43) Pub. Date: Nov. 29, 2012

(54) DOCKING SYSTEM FOR A CPAP MACHINE

(75) Inventor: Michael G. Lalonde, Alpharetta, GA (US)

(73) Assignee: DESHUM MEDICAL, LLC, Cambridge, MA (US)

(21) Appl. No.: 13/450,614

(22) Filed: Apr. 19, 2012

Related U.S. Application Data
(63) Continuation-in-part of application No. PCT/US2010/053370, filed on Oct. 20, 2010.
(60) Provisional application No. 61/253,500, filed on Oct. 20, 2009.
(60) Provisional application No. 61/288,290, filed on Dec. 19, 2009.
(60) Provisional application No. 61/301,151, filed on Feb. 3, 2010.
(60) Provisional application No. 61/560,271, filed on Nov. 15, 2011.

(51) Int. Cl. A61M 16/00; A61M 11/00; A61M 16/16

(52) U.S. Cl. 128/200.16; 128/202.27

(57) ABSTRACT

A docking station holds a flow generator of a CPAP system. The docking station has many purposes including having an acoustic chamber to reduce the inherent acoustic noise including from the intake of the flow generator that the user and other hear. The docking station has additional features including humidifying and intake air filtering.

### CROSS-REFERENCE TO RELATED APPLICATIONS

This application is a continuation-in-part of PCT Application PCT/US2010/053370 filed on Oct. 20, 2010 which claims the benefit of U.S. Patent Application 61/253,500 filed on Oct. 20, 2009, U.S. Patent Application 61/288,290 filed on Dec. 19, 2009, and U.S. Patent Application 61/301,151 filed on Feb. 3, 2010, and this application claims the benefit of U.S. Patent Application 61/560,271 filed on Nov. 15, 2011, which are incorporated herein by reference.

### FIELD OF THE INVENTION

The present invention relates to a continuous positive airway pressure (CPAP) machine and more particularly to a docking station that receives the flow generator.

### BACKGROUND OF THE INVENTION

Sleep apnea syndrome afflicts an estimated 1% to 5% of the general population and is due to episodic upper airway obstruction during sleep. Those afflicted with sleep apnea experience sleep fragmentation and intermittent, complete, or nearly complete cessation of ventilation during sleep with potentially severe degrees of oxyhemoglobin desaturation.

Although details of the pathogenesis of upper airway obstruction in sleep apnea patients have not been fully defined, it is generally accepted that the mechanism includes either anatomic or functional abnormalities of the upper airway which result in increased air flow resistance. Such abnormalities may include narrowing of the upper airway due to suction forces involved during inspiration, the effect of gravity pulling the tongue back to oppose the pharyngeal wall, and/or insufficient muscle tone in the upper airway dilator muscles. It has also been hypothesized that a mechanism responsible for the known association between obesity and sleep apnea is excessive soft tissue in the anterior and lateral neck which applies sufficient pressure on internal structures to narrow the airway.

Recent work in the treatment of sleep apnea has included the use of continuous positive airway pressure (CPAP) to maintain the airway of the patient in a continuously open state during sleep. Unfortunately, the statistics on CPAP non-compliance are startling. There are numerous reasons for non-compliance including the noise and discomfort of exhaling against a positive air pressure.

### SUMMARY OF THE INVENTION

It has been recognized that conventional CPAP (continuous positive airway pressure) machines to treat apnea provide a positive pressure to the user when the unit is turned on. The flow generator in compressing the air has inherent acoustic noise including at the intake. The system has a docking station that includes an acoustic suppression baffle to reduce the noise. The docking station in addition can have additional features such an air intake filter and humidifier for conditioning the air. A humidifier has a warming element and water reservoir and conduction means to the breathable gas output to supply humidity. The humidifier is controlled by humidity control and/or by the remote control. The docking station also has an internal power supply and power management.

In an embodiment of a docking station for a flow generator of a continuous positive airway pressure (CPAP) system, the docking station has a housing having an insertion cavity adapted to receive the flow generator. The housing defines an input air flow path having a breathable gas outlet for communicating air to an inlet on the flow generator. The flow path includes an acoustic chamber for reducing noises.

In an embodiment, the docking station has a power management system including a pair of power contacts carried on the housing defining the insertion cavity for communicating energy to the flow generator.

In an embodiment, the docking station has a humidifier having a flow path. The humidifier has a reservoir for water, a mechanism to create vapor, and an air mixing chamber for mixing the humidified air with air from the input air flow path prior to the breathable gas outlet.

In an embodiment, the mechanism to create vapor is a heater. In an embodiment, the mechanism to create vapor is an ultrasonic device.

In an embodiment, the docking station has a cover adapted to substantially cover the flow generator. The cover defines an enclosure which carries foam sound absorbing.

In an embodiment, the cover is pivotably mounted to the base for moving between an open position granting access to the insertion cavity and a closed position overlying the insertion cavity.

In an embodiment, the acoustic chamber has baffle walls with sound absorbing acoustic foam material.

In an embodiment, the CPAP system has a flow generator and a docking station. The flow generator has a breathable gas intake and a hose interface. The flow generator has a compressor for pressurizing the gas received through the breathable gas intake and exhaling through the hose interface. The docking station has a housing having an outer enclosure wall and an insertion cavity to receive the flow generator. The housing of the docking station defines an input air flow path having a breathable gas outlet for communicating air to the inlet on the flow generator. The flow path includes an acoustic chamber for reducing noises. The housing defines a cavity between the insertion cavity and the outer enclosure for a hose from the hose interface of the flow generator.

In an embodiment, the docking station further comprises a latch having a latch hook for engaging the flow generator to retain the flow generator in the insertion cavity of the docking station.

In an embodiment, the flow generator has a battery. The battery is chargeable by the power management system.

These aspects of the invention are not meant to be exclusive and other features, aspects, and advantages of the present invention will be readily apparent to those of ordinary skill in the art when read in conjunction with the following description, appended claims, and accompanying drawings.

### BRIEF DESCRIPTION OF THE DRAWINGS

The foregoing and other objects, features, and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1A is a side view of a detachable flow generator coupled to a docking station;

FIG. 1B is a perspective view of the detachable flow generator coupled to the docking station with a hose extending to a mask;

FIG. 2 is a perspective view of an alternative docking station with a CPAP flow generator and a remote exploded away;

FIG. 3 is sectional view of the docking station taken along line 3-3 in FIG. 2;

FIG. 4 is an enlarged view of a portion of the docking station showing the latch;

FIG. 5 is a sectional view of the docking station taken along line 5-5 in FIG. 2;

FIG. 6 is rear view of the docking station;

FIG. 7 is a flow chart illustrating the logical operation of the basic functions of the system;

FIG. 8 is a perspective view of a cover for the docking station; and

FIG. 9 is a sectional view of the cover taken along line 9-9 in FIG. 8.

### DETAILED DESCRIPTION OF THE INVENTION

A system for delivering pressurized gas to the airway of a patient; the system has a gas flow generator for providing a flow of gas and a mask for the delivery of the gas flow to an airway of a patient. The flow generator in compressing the air has inherent acoustic noise including at the intake. The system has a docking station that includes an acoustic suppression baffle to reduce the noise. The docking station in addition can have additional features such an air intake filter and humidifier for conditioning the air. A humidifier has a warming element and water reservoir and conduction means to the breathable gas output to supply humidity. The humidifier is controlled by the humidity control and/or by the remote control. The docking station also has an internal power supply and power management.

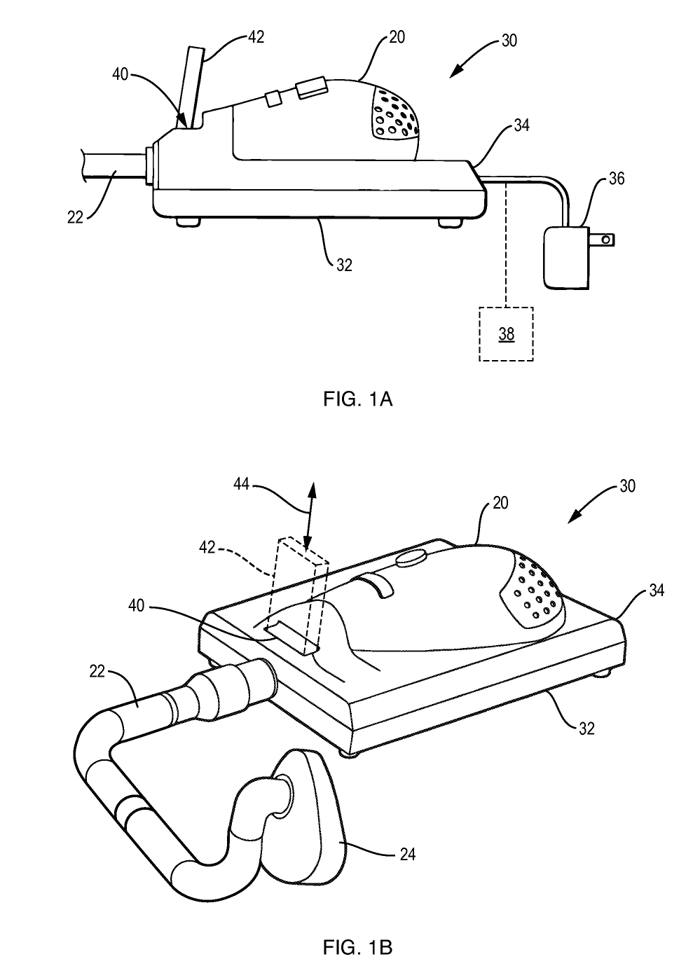

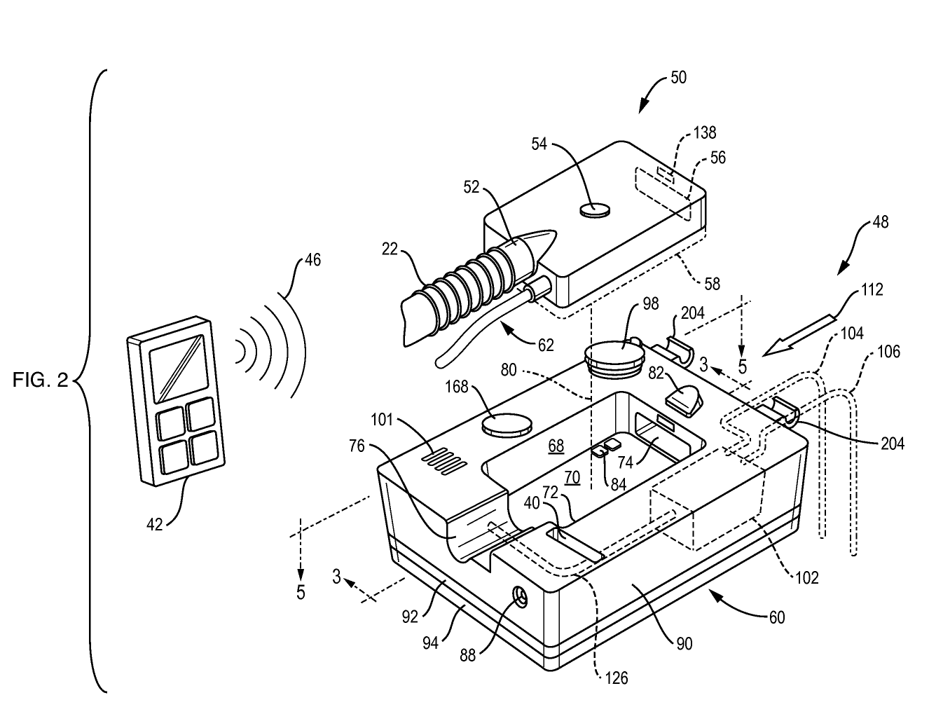

Referring to FIG. 1A, a side view of a flow generator 20 connected to a docking station 30, also referred to as a stationary base unit is shown. FIG. 1B shows a perspective view of the flow generator 20 in the docking station 30 and a hose 22 connected to a mask 24. The separable flow generator 20 can be coupled into the docking station 30 in order to supply gas to a mask unit 24 as shown in FIG. 1B. In this way, the stationary base unit, the docking station 30, may be a relatively simple device, and all of the components are included in the separable flow generator 20 to be used in connect directly via the hose 22 to the mask 24; the docking station 30 provides power.

The docking station 30, sometimes referred to as a stationary base unit, includes a battery 32 that is dedicated or rechargeable. Additionally, the docking station 30 includes a connection on a base 34 for coupling to an AC adaptor 36 or a power source 38, which is shown in phantom in FIG. 1A which can include batteries, a fuel cell for power, or an automobile DC circuit adaptor.

The docking station 30 has a docking receptacle 40 that receives a remote control 42, which may be charged in the docking receptacle 40. Typically, the remote control 42 may be used to control the flow generator 20. The remote control 42 is insertable and removable from the docking receptacle 40 as shown by the arrows 44. In one embodiment, the remote control may be charged and or docked in a separate dedicated docking station.

The hose 22 may include an adapter to attach to the detachable mask 24, thus permitting the reuse of the detached mask 24 when the flow generator 30 is remotely docked.

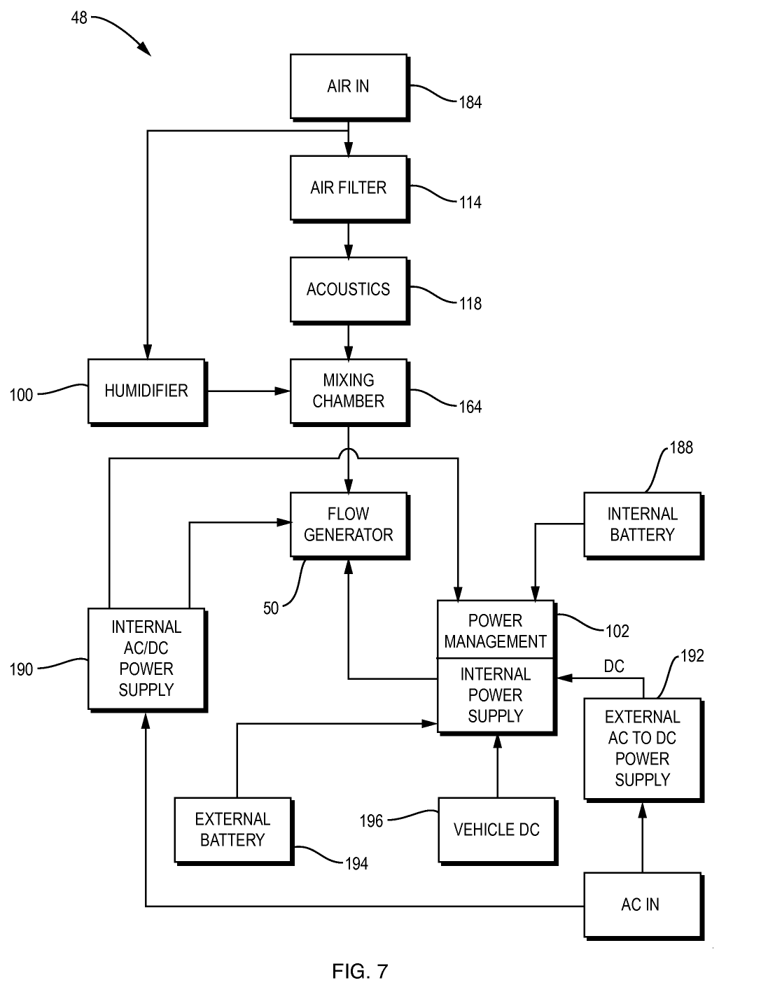

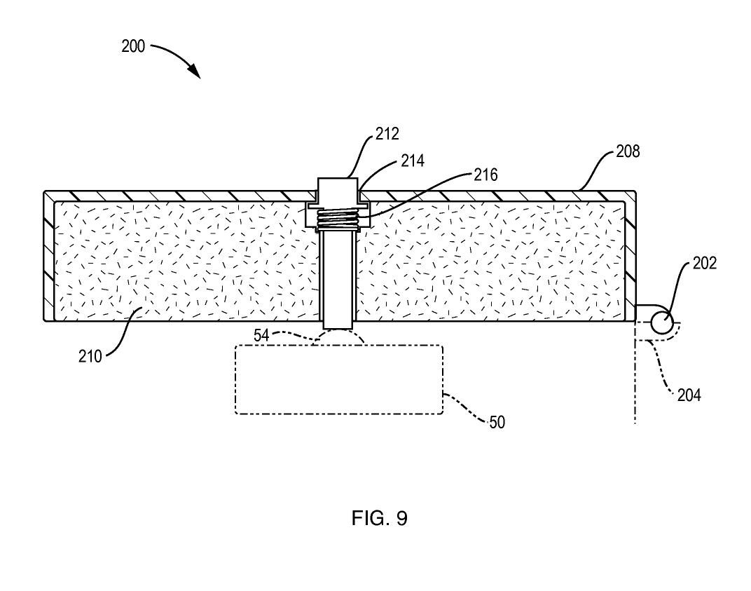

The flow generator 20 shown in FIGS. 1A and 1B is configured to connect directly to a mask. FIGS. 7A and 9 of U.S. patent application Ser. No. ______ which was filed on ______ as the National Phase of PCT Application PCT/US2010/053370 filed on Oct. 20, 2010 shows the flow generator attached to the mask. While the docking station 30 shown in FIGS. 1A and 1B.

Referring to FIG. 2, a perspective view of an alternative docking station 60 with a CPAP flow generator 50 and the remote control 42 exploded away is shown. The CPAP system 48 has the flow generator 50, the hose 22, and a mask 24. The docking station 60 receives the flow generator 50. The flow generator 50 has a hose interface 52, an operation control button 54, and a breathable gas intake 56, shown in hidden line. The flow generator 50 has a compressor for taking ambient air from the breathable gas intake 56 and creating pressurized air flow. The pressure range desired can vary, but generally falls in the range of between 4 and 20 centimeters of water. The range of the system 20 can extend even higher from 0 centimeters of water to 30 or 50 centimeters of water. The average user/patient typically is treated by a pressure of between 6 and 14 centimeters of water. A typical user utilizes an air flow rate of 20 to 60 liters of air per minute.

The flow generator 50 has an optional attachable battery 58. The system 48 has a DC power cord 62 and AC to DC power supply (not shown). The remote control 42 communicates through a wireless signal 46.

The docking station 60 of the system 48 has a plurality of walls 68 and a base 70 defining an insertion cavity 72 for receiving the flow generator 50. One of the walls 68 of the insertion cavity 72 has a breathable gas outlet 74. One of the walls 68 has a hose and power cord cavity 76.

Air is connected to the flow generator 50 through the breathable gas output 74 which corresponds to the breathable gas intake 56. The hose 22 and the power cord 62 are seated and pass through the hose and power cord cavity 76.

The flow generator 50 is inserted into the insertion cavity 72 by way of an insertion/removal path 80 and is secured with the latch 82. The flow generator 50 is connected to power by way of a pair of power contacts 84. Power from the contacts 84 may be used to power the flow generator 50 directly and/or charge the optional attachable battery 58 when present.

The power cord 62 can attach to either the flow generator 50 AC to DC power supply or connect to a DC power receptacle 88 on the docking station 60. The docking station 60 has a plurality of enclosure walls 90 including the wall that has the hose and power cord cavity 76 and the DC power receptacle 88. The docking station 60 has a stabilizing weight 92 and a slip-resistant grip pad 94.

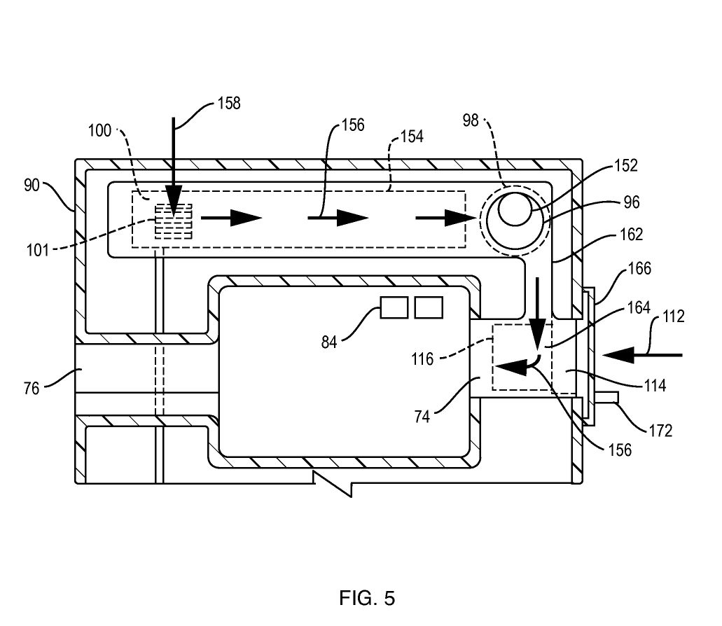

The docking station 60 has a water inlet 96, as seen in FIG. 5 that is covered by a cap 98 which communicates water to the humidifier 100 as best seen in FIG. 5. The air is drawn through a vent 101 to the humidifier 100.

Still referring to FIG. 2, the system 48 has an internal power supply and power management 102 provides power to the flow generator, the humidifier 100, as seen in FIG. 5 and a remote control dock or docking receptacle 40. The system 48 has an AC power cord 104 which supplies power to the internal power supply 102 and is acted upon by power management. The docking station 60 has a DC power cord 106 that can supply power to the power supply 102 and is also acted upon by the power management circuitry 102. The DC power cord 106 attaches to any one of the DC power sources including an external AC to DC power supply 192, an external battery 194, an vehicle DC power source 196, and other power sources as represented in FIG. 7 and known to those skilled in the art. Power management determines which power input to permit. For example, should DC power 106 be connected; power management would decide to override AC power 104 if applied after the DC power was connected. Conversely, if AC power is connected first, then power management would override the DC power if connected afterwards.

In an embodiment, the walls of the insertion cavity 72 possess a gripping material such as silicone or rubber or similar to grip the flow generator 50 without the need of a latch 82. The shape of the gripping material may also encourage further adhesion. Alternatively, the walls 68 of the insertion cavity 72 possess a gripping material that is used in conjunction with the latch 82.

In an embodiment, system 48 has a remote control 42. The remote control 42 may be powered by chargeable, non-chargeable batteries or wired. The remote control 42 communicates to the system wired or wirelessly. The remote control 42 may also be compatible with the flow generator 20. The remote control 42 can dock into a remote control dock 40 where power and/or communications are supplied. The dock 40 may also provide just a cavity in which to store and display the remote.

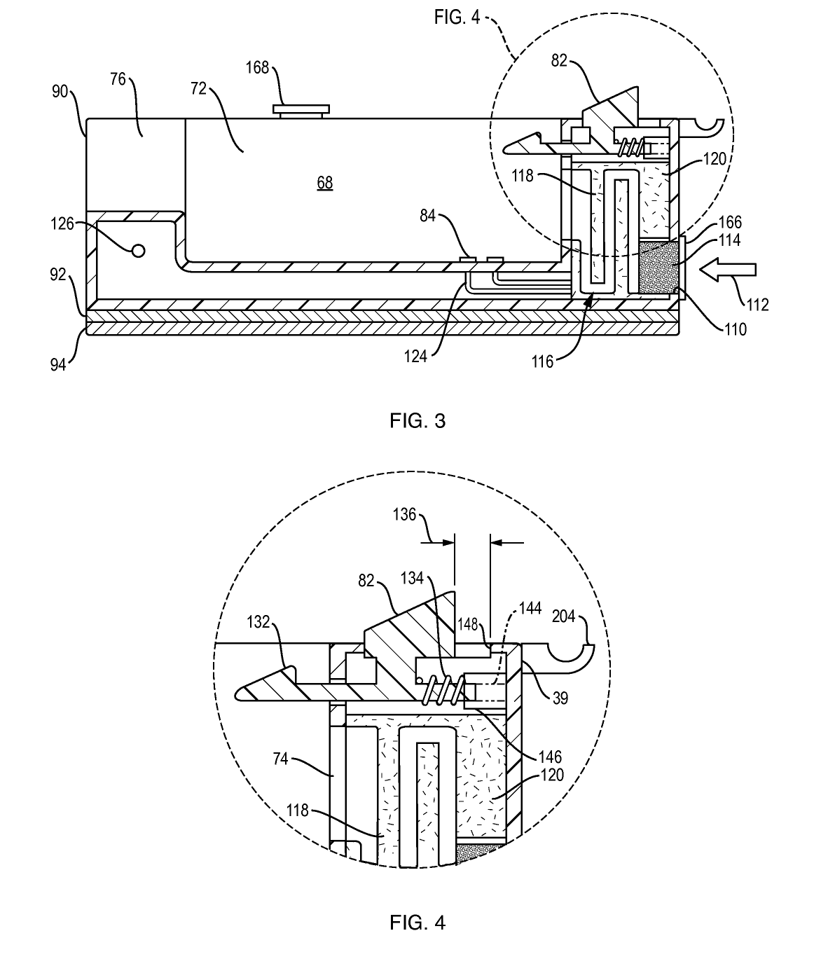

Referring to FIG. 3, a sectional view of taken along line 3-3 in FIG. 2 of the docking station 60. The docking station 60 has an air take opening 110 through which air is drawn as represented by an air path arrow 112. The air passes through an air intake filter 114 before mixing with humidified air supplied by the humidifier 100, as best seen in FIG. 5. The intake filter 114 can be fabricated from bio-compatible materials known to the industry. The filter 114 can also be a HEPA-filtration type. The filter 114 prevents contamination of an acoustic chamber 116 and adds further filtration to the flow generator 50 and resulting patient air-way. The air continues to pass through the acoustic suppression chamber 116 in the docking station 60. The chamber 116 is divided by a plurality of baffle walls 118. When the flow generator 50 is operating it emanates acoustic energy. The baffle walls 118 are formed out of an absorbed acoustic foam material 120 which constitutes the acoustic chamber 116. The convoluted path of the acoustic chamber 116 is disposed in a way to optimally absorb acoustic energy. There is a sealable connection between the intake 56, as seen in hidden line in FIG. 2, of the flow generator 50 and the gas outlet 74 which prevents air leakage and acoustic noise. The power contacts 84 are connected to the power supply 102, as seen in FIG. 2, via a power conductor 124. A power conductor 126 provides power from the power supply 102 to the humidifier 100.

In one embodiment, the acoustic chamber 116 can be constructed of a more solid material such as high durometer plastic such as PVC or similar material. There may also be a combination of a softer material such as foam 120 and harder material.

In one embodiment, the acoustic chamber 116 may be replaced with an active or passive noise cancelation method. Active noise cancelation employs a means of producing the same or near same frequency of the sound desired to suppress and is generated to cancel out the sound. Passive noise cancellation oscillates at the same sound energy to reflect back on itself resulting in a net cancelation.

In one embodiment, the acoustic chamber 116 is larger than depicted in FIG. 3. The chamber can occupy a larger space in any one of several areas within the system enclosure where space permits. Larger chambers permit even greater effectiveness in suppressing acoustic noise.

In one embodiment the air and acoustic seal between the breathable gas outlet 74 of the docking station 30 and the breathable gas intake 56 of the flow generator 50 is achieved instead by a secondary seal in and around the perimeter of the insertion cavity 72. It is recognized that both the perimeter seal in the insertion cavity 72 and the seal between the outlet 74 and the intake 56 are employed. In addition, the perimeter seal in the insertion cavity 74 can assist in retaining the flow generator 50 with friction.

Referring to FIG. 4, an enlarged view of a portion of the docking station 60 showing the latch 82 is shown. The latch 82 has a latch hook 132 and a spring 134. When latch 82 is moved in the direction of travel 136, the spring 134 compresses and creates tension on the latch 82.

The movement of the latch hook 132 and compression of the spring 134 can occur by the insertion of the flow generator 50, as seen in FIG. 2, into the insertion cavity 72. The bottom of the flow generator 50 engages the sloped top of the latch hook 132 and forces the latch hook 132 in the direction of travel 136. Alternatively, the movement of the latch hook 132 and compression of the spring 134 can occur by the sliding of the exposed part of the latch 82 with a finger to permit easy insertion of the flow generator 50.

The flow generator 50 is secured in the insertion cavity 72 by the latch hook 132 being inserted into a hook receiving slot 138, as seen in hidden line in FIG. 2, of the flow generator 50. The bottom of the latch hook 132 engages the bottom wall of the hook receiving slot 138 to retain the flow generator 50 in the insertion cavity 72 aligning electrical contacts with the power contacts 84 on the base 70 of the insertion cavity 72.

The flow generator 50 is released by sliding the exposed part of the latch 82 with a finger to compress the spring 134 extracting the latch hook 132 from the hook receiving slot 138 and permitting removal of the flow generator 50 in the direction of the path 80 as seen in FIG. 2. The latch 82 has a shaft 142 that is supported by a shaft hole 144 in a shaft receiver 146. The travel of the latch 82 is limited by the size of a slot 148 through which the exposed part of the latch 82 projects.

Referring to FIG. 5, a sectional view of the docking station taken along line 5-5 in FIG. 2 is shown. The humidifier 100 of the docking station 30 provides humidification to air drawn in by the flow generator 50. The humidifier 100 is filled with water through the water inlet 96 covered by the cap 98, as seen in FIG. 2. The water passes through the water inlet 96 to a trough water inlet 152 into a humidifier reservoir 154. The heating element beneath the humidifier reservoir 154 is connected to the power conductor 126. Water is heated by the heating element heating the water resulting in some of the water to evaporation creating a vapor. Water vapors from the evaporation flow are mixed with air that is in a pass-over air path as represented by arrows 156. The air that is humidified is drawn into the humidifier vent 101 as represented by the air path arrow 158. The humidifier 100 within the docking station 30 has a humidifier exhaust channel 162 that opens into an air mixing chamber 164 and mixes with the incoming air as represented by the arrow 112. The incoming air passes through an air intake cover 166 and through the air intake filter 114 and the acoustic suppression chamber 116. Un-humidified air and vapors mix and result in humidified air transmitted through the breathable gas outlet 74 and drawn into the breathable gas intake 56 of the flow generator 50. The heat created by the heating element and the level of resulting vapor is controlled by an element controller which is further controlled by a control knob 168, as seen in FIG. 2. The element controller is contained within the power management 102. The docking station 30 has an auxiliary gas port 172 that can be connected to various breathable gases including oxygen, gasified medications, and others known by those skilled in the art.

In one embodiment, water vapors are created by an ultrasonic means instead of heated evaporation. High frequency modulation of a surface to which the water is exposed atomizes the water to produce vapor.

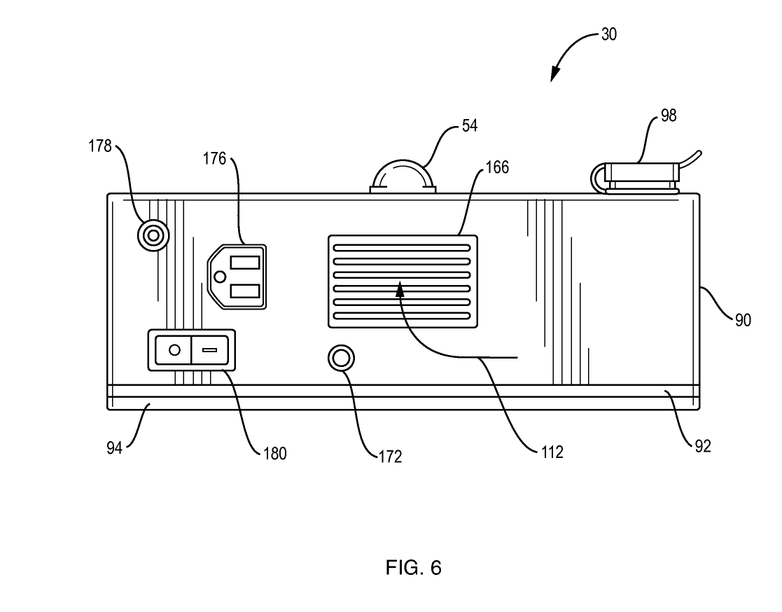

Referring to FIG. 6, a rear view of the docking station 30 is shown. The system 48 has power inputs on the docking station 30 of both an AC receptacle 176 and DC receptacle 178. Power to the system is enabled and disabled by a power switch 180. Alternatively to the power switch 180, the system 48 is powered by merely attaching to a power source. The system 48 goes into standby mode until activated by the activation of the flow generator 50 using the operation control button 54.

Referring to FIG. 7, a flow chart illustrates the logical operation of basic functions of the system 48. Air is aspirated in the docking system 30 in two different areas, into the air filter 114 and the humidifier 100. Air passing through the air filter 114 passes through the acoustic suppression 116 to the humidity mixer 164. The humidifier 100 produces water vapors which are ingested into the humidity mixer 164 along with un-humidified air. The humidity mixer 100 mixes the two and passes the humidified air to the flow generator 50. The system 48 is powered by either of an internal battery 188, an internal power supply 190, an external power supply 192, an external battery 194, or a vehicle's DC 196. The AC power is supplied to the external power supply 192 and the internal power supply 190. The power management and internal power supply 102 manage all power activities of the system 48.

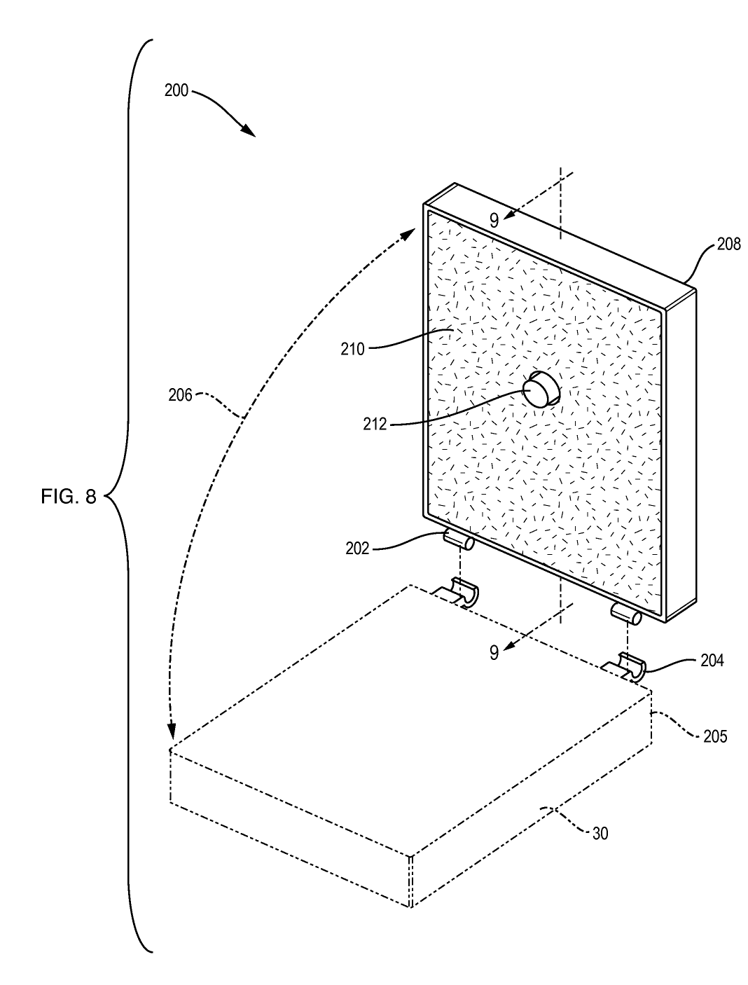

Referring to FIG. 8, a perspective view of a cover 200 for the docking station 30 is shown. The cover or lid 200 has a pair of hinge parts 202 that are received by a pair of hinge receivers 204 carried by a base 204 of the docking station 50 shown in hidden line. When the cover 200 is attached to the base 204, the cover 200 follows an open/closure path 206 to allow access to the flow generator 50 and to cover the flow generator 50 to reduce acoustic noise.

The cover or lid 200 defines an enclosure 208 that is hollow and is filled with foam 210. The cover 200 has a translation button 212 which is located to match the control button 54 on the flow generator 50.

Referring to FIG. 9, a sectional view of the cover 200 taken along line 9-9 in FIG. 8 is shown. The cover 200 has a hole 214 through which the translation button 212 extends. The translation button 212 protrudes above the surface of the cover 200. The translation button 212 engages the operable control button 54 of the flow generator 50. When the button 212 is depressed by the user, the button 212 will move towards the flow generator which depresses the button 52. The button 212 retracts by release of compression of a spring 214 and/or is assisted by movement of the operation control button 54 on the flow generator 50.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

It is recognized that the docking station 50 including the cover 200 can have controls. The controls may include a multi-function interface. The controls may include a user display. The control may be used in lieu of remote control or the remote may be used in lieu of the controls.

The abbreviation CPAP stands for continuous positive air pressure which in generic terms is a method of noninvasive or invasive ventilation assisted by a flow of air delivered at a positive pressure throughout the respiratory cycle. It is performed for patients who can initiate their own respirations but who are not able to maintain adequate arterial oxygen levels without assistance. Sometimes the word “continuous” is replaced with the “constant.” For the purpose of this patent, constant positive airway pressure is referred to as mono-level CPAP. CPAP can be in various modes including mono-level CPAP, Bi-level CPAP, Auto-PAP, Servo-ventilation, and ramping.

It is recognize that the docking station 50 can take other forms. Provisional application U.S. Patent Application 61/560,271 filed on Nov. 15, 2011, which is incorporated herein by reference, describes some other docking stations.

It is recognized that besides humidification and noise reduction, the docking station allows the transformation of a portable flow generator of the CPAP into a stationary device when desired. This provides stationary means to secure the CPAP device when installed, so that it remains in a pre-determined place with minimal or no movement when there is tugging on the hose.

The docking station can be placed near the patient on a stationary surface such as a night stand or dresser. It is recognized that it can be placed in another location such as on the bed, on a lounge chair, or on a couch near the user.

In addition to the benefits mentioned above including intake air filtering and acoustic suppression, other benefits include vibration suppression and interfacing the flow generator to a data link.

What is claimed:

1. A docking station for a flow generator of a continuous positive airway pressure (CPAP) system, the docking station comprising:
a housing having an insertion cavity adapted to receive the flow generator; and
the housing defining an input air flow path having a breathable gas outlet for communicating air to an inlet on the flow generator, the flow path including an acoustic chamber for reducing noises.

2. A docking station of claim 1 further comprises a power management system including a pair of power contacts carried on the housing defining the insertion cavity for communicating energy to the flow generator.

3. A docking station of claim 1 further comprising a humidifier having a flow path, a reservoir for water, a mechanism to create vapor, and an air mixing chamber for mixing the humidified air with air from the input air flow path prior to the breathable gas outlet.

4. A docking station of claim 3 wherein the mechanism to create vapor is a heater.

5. A docking station of claim 3 wherein the mechanism to create vapor is an ultrasonic device.

6. A docking station of claim 1 wherein the housing defines a base and further comprises a cover adapted to substantially cover the flow generator, the cover defining an enclosure, and the docking station having foam carried within the enclosure of the cover.

7. A docking station of claim 6 wherein the cover is pivotably mounted to the base for moving between an open position granting access to the insertion cavity and a closed position overlying the insertion cavity.

8. A docking station of claim 1 wherein the acoustic chamber has baffle walls with acoustic absorbing foam material.

9. A CPAP system comprising:
a flow generator having a breathable gas intake and a hose interface, the flow generator having a compressor for pressurizing the gas received through the breathable gas intake and exhaling through the hose interface; and
a docking station having a housing having an outer enclosure wall and an insertion cavity to receive the flow generator,
the housing of the docking station defining an input air flow path having a breathable gas outlet for communicating air to the inlet on the flow generator, the flow path including an acoustic chamber for reducing noises; and
the housing defining a cavity between the insertion cavity and the outer enclosure for a hose from the hose interface of the flow generator.

10. A CPAP system of claim 9 wherein the docking station further comprises a power management system including a pair of power contacts carried on the hosing defining the insertion cavity for communicating energy to the flow generator.

11. A CPAP system of claim 9 wherein the docking station further comprises a humidifier having a flow path, a reservoir for water, a mechanism to create vapor, and an air mixing chamber for mixing the humidified air with air from the input air flow path prior to the breathable gas outlet.

12. A CPAP system of claim 11 wherein the mechanism to create vapor is a heater.

13. A CPAP system of claim 11 wherein the mechanism to create vapor is an ultrasonic device.

14. A CPAP system of claim 9 wherein the housing of the docking station defines a base and further comprises a cover adapted to substantially cover the flow generator, the cover defining an enclosure, and the docking station having foam carried within the enclosure of the cover.

15. A CPAP system of claim 14 wherein the cover is pivotably mounted to the base for moving between an open position granting access to the insertion cavity and a closed position overlying the insertion cavity.

16. A CPAP system of claim 9 wherein the docking station further comprises a latch having a latch hook for engaging the flow generator to retain the flow generator in the insertion cavity of the docking station.

17. A CPAP system of claim 9 wherein the acoustic chamber of the docking station has baffle walls with acoustic absorbing foam material.

18. A CPAP system of claim 10 wherein the flow generator has a battery and the battery is chargeable by the power management system.
